SEQUENCE LISTING (12 sequences)

SEQ ID NO: 1 - DNA, 36 bp, Synechocystis PCC 6803
gagagagagagactcgagtacggggtgattagtatt

SEQ ID NO: 2 - DNA, 35 bp, Synechocystis PCC 6803
gagagagagagaccgcggacctccattgaggtcta

SEQ ID NO: 3 - DNA, 38 bp, Synechocystis PCC 6803
gagagagagactcgagttggttataattccttatgtat

SEQ ID NO: 4 - DNA, 33 bp, Synechocystis PCC 6803
gagagagagaggtaccagcgttccagtggatat

SEQ ID NO: 5 - DNA, 34 bp, Synechocystis PCC 6803
tatatatataccgcgggtagagttgcgagggcga

SEQ ID NO: 6 - DNA, 25 bp, Synechocystis PCC 6803
taattccttggtgtaatgccaactg

SEQ ID NO: 7 - DNA, 20 bp, Chlamydomonas reinhardtii
tgtcacgccacggtttgaac

SEQ ID NO: 8 - DNA, 22 bp, Chlamydomonas reinhardtii
tcacgtttgctggtgttgctac

SEQ ID NO: 9 - DNA, 53 bp, Chlamydomonas reinhardtii
acaaacaacattacaattactatttacaattacaatggccaggtgagtcgacg

SEQ ID NO: 10 - DNA, 55 bp, Chlamydomonas reinhardtii
tgtttgttgttgttggtaattgttgtaaaaatactaagatgttgagtgacttctc

SEQ ID NO: 11 - DNA, 62 bp, Chlamydomonas reinhardtii
atcagcgaggaggacctgaacagcgagaaggacgagctgtaaactggcagcccgccaacaag

SEQ ID NO: 12 - DNA, 50 bp, Chlamydomonas reinhardtii
cagcttctgctcggcggccgccaggatccaggcctccaggccctgcaatg

(19) United States
(12) Patent Application Publication
(10) Pub. No.: US 2005/0282247 A1
(43) Pub. Date: Dec. 22, 2005

(54) Recombinant single chain antibody in heterotrophic algae for pathogen control and use

(76) Inventors: Madeline Chang Sun Wu, Hong Kong (HK); Sally Ka Kan Mak, Hong Kong (HK); Ken Wan Keung Lau, Hong Kong (HK); Jianping Ren, Hong Kong (HK)

Correspondence Address: BAKER & MCKENZIE;PATENT DEPARTMENT, 2001 ROSS AVENUE, SUITE 2300, DALLAS, TX, 75201, US

(21) Appl. No.: 10/144,557

(22) Filed: May 13, 2002

(51) Int. Cl. A61K 39/40; C12N 1/12; C07H 21/04; C12P 21/02; C12N 15/74; C07K 16/12

(52) U.S. Cl. 435/69.1; 435/257.2; 435/320.1; 530/388.5; 536/23.53; 424/164.1

(57) ABSTRACT

Expression of functionally active recombinant single chain antibody in prokaryotic and eukaryotic heterotrophic algae cells for pathogen treatment or control in aquaculture and agriculture applications is disclosed; and subsequent modification of the strains to generate transgenic algae propagated in defined conditions.

### REFERENCE TO SEQUENCE LISTING

Two copies of the “Sequence Listing” in computer readable form in compliance with 37 C.F.R. §1.821 to 1.825 are enclosed. The sequence listing information recorded in computer readable form is identical to the written on paper sequence listing, and incorporated herein by reference.

### BACKGROUND OF THE INVENTION

The present invention relates to a high level expression of a functional single chain antibody in prokaryotic and eukaryotic algae cells and, more particularly to subsequent modification of the strains to generate transgenic algae propagated in defined conditions, and its use in environmental applications.

Aquaculture is a rapidly expanding industry. The demand for aquaculture products is rising continuously because the wild population for many species are declining. For example, aquaculture species, such as prawn, abalone or the like, are susceptible to bacterial, viral and parasitic diseases. In the dense culture situation used in aquaculture, the infections often transmit rapidly, inflict massive mortality, and cause massive economic loss. Once there is an outbreak in disease, the pathogen may persist for a long time despite attempts to remove them by expensive pond drying, sun exposure, and the installation of complicated water treatment systems. The prior art uses antibiotics, chemicals to control pathogens or both. Both measures are not effective for all types of pathogen and further they cannot differentiate the pathogens from the benign species. The removal of benign species often makes the aquaculture species more susceptible to other infections. Bacteria pathogens, such as Vibrio spp, have developed antibiotic-resistant strains. Some bacteria species that infect aquatic species, such as Vibrio cholerae, also can cause diseases in humans. To prevent the spread of antibiotic-resistant pathogens, many countries have banned the import of any farmed species that have been exposed to antibiotics and highly sensitive detection methods for antibiotics were developed. The accumulation of many potentially carcinogenic chemicals in the cultured species and the environment also causes increased concern and increased regulatory control.

Moreover, a recombinant immune system is a feasible but expensive method for disease control. Immunization of animals with a particular antigen to produce specific antibodies for disease control is a well known process in humans. The immune responses for most aquaculture species are not clear, and field data clearly showed its insufficiency to deal with the challenges of aquaculture conditions. Farming animals, such as cow, swine and fowl (e.g., chicken) have immune systems, but the animals' own immune systems usually do not respond quick enough to prevent the massive disease outbreak. In theory, the monoclonal antibodies for a targeted pathogen can be produced using the specific antigen of that pathogen and the immune response of mice. A single chain recombinant antibody retains full antigen-binding activity and a recombinant gene coding for such antibody can be prepared. The single chain antibodies consist of variable light chain domain and heavy chain domains of an antibody molecule fused by a flexible peptide linker as described in U.S. Pat. No. 5,863,765, the teachings of which are incorporated herein by reference. However, they retain full antigen-binding activity but lack the complex assembly requirements and are more suitable for expression in other organisms. Many single chain antibodies have proven record for controlling human diseases over, 100 antibodies therapeutics are currently in clinical trials for cancer, viral, autoimmune, and other diseases as described by E. T. Boder, E. T., et al., Proc. National Academy of Science, USA 97, 10701-10705 (2000), the teachings of which are incorporated herein by reference.

In addition, recombinant vectors encoding IG like domains are disclosed in U.S. Pat. No. 6,165,745. Nonetheless, both the high cost and the lack of suitable vector to carry the antibody to the target disease sites prohibit the usage of single chain antibody for aquaculture and agriculture. There is a need for a high level expression of a functional gene in algal cells. The present invention meets this need.

### SUMMARY OF THE INVENTION

One embodiment of the present invention is a process for treating pathogen comprising, cloning and expressing a recombinant single chain antibody in heterotrophic algal cells selected from the group consisting of prokaryotic strain and eukaryotic strain.

Another embodiment of the present invention is an insertion site and native promoter combination suitable for foreign gene integration in heterotrophic algal strains.

Yet another embodiment of the present invention is genetically engineered DNA molecules that facilitate the site-specific integration and expression of single chain antibody genes in the prokaryotic alga, preferably Synechocystis PCC 6803, and eukaryotic alga, preferably Chlamydomonas reinhardtii.

The present invention provides a viable remedy to control and/or treat pathogens for aquaculture and agriculture species. Engineered algal strains that can express high level of functional single chain antibody were constructed. Since the production cost for the algae is low, the transgenic algal species cannot propagate in nature, and they are non-toxic. They can be either used as feed supplement or applied directly in the field for pathogen control or both. This invention can also offer a measure to include proper single chain antibody for pathogenic viruses in the food supplement to control the outbreak of viral diseases such as, for example, the foot and mouth disease and bird-flu. Therefore, the present invention achieves economically safe and cost effective measures for diagnosis, control and treatment of epidemic diseases caused by a wide spectrum of pathogens including viruses, bacteria, fungi, protozoa and the like.

Details of one or more embodiments of the invention are set forth in the description below. These embodiments are for illustrative purposes only and the principle of invention can be implemented in other embodiments. Other features and advantages of this invention will become apparent from the following description.

### BRIEF DESCRIPTION OF THE DRAWINGS

FIG. 1 illustrates the growth rate of mutant with rfbJ gene interrupted under different conditions compared with that of the wild type of Synechocystis PCC 6803.

FIG. 2 is a diagram of the recombinant DNA molecule used for Synechocystis PCC 6803 transformation.

FIG. 3 illustrates a diagram of the recombinant DNA molecule used for transformation of Chlamydomonas reinhardtii.

FIG. 4 is a scanned image of the Southern blot result of the transgenic alga that contains a single insert of Sca at the rfbJ target site. The DNA was digested with Bst N1.

FIG. 5a. The RT-PCR result shows the transcription of introduced Sca gene in Synechocystis PCC 6803.

FIG. 5b. The Western blot result shows the expression of Sca in different growth condition.

FIG. 6 illustrates the specificity of Sca expressed in algal cells.

FIG. 7 illustrates the relative binding affinities for progesterone conjugates.

FIG. 8 illustrates the structure of the progesterone conjugates used for specificity analysis.

### DETAILED DESCRIPTION OF THE INVENTION

A high-level expression of functional foreign gene in algal cells has been achieved by the present invention. More specifically, a significant improvement of the transformation and selection processes for the prokaryotic alga, preferably Synechocystis PCC 6803 and the eukaryotic alga, preferably Chlamydomonas reinhardtii has been achieved. Conditions for high frequency homologous recombination have been established previously and a portion of the details was published (Tang et al. 1995). Tang, D. K. H., Qiao, S.-Y, and Wu, M., (1995) Biochemistry and Molecular Biology International 36, 1025-1035, the teachings of which are incorporated herein by reference. Recombinant DNA technology is used to clone DNA sequences encoding the protein or proteins to be used as immunogens into a prokaryotic or eukaryotic expression vector.

Selection of Proper Insertion Sites:

Random gene-knock out using the Km gene for Synechocystis PCC 6803 and the ble gene for Chlamydomonas reinhardtii as selection markers may be carried out. Mutants with normal or higher growth rate may be selected for TAIL-PCR using primers of SEQ ID NO: 11 and SEQ ID NO: 12 for Synechocystis PCC 6803 and primers of SEQ ID NO: 6 and SEQ ID NO, 7 for Chlamydomonas reinhardtii. Southern blot analyses were used to determine the insertion copy number. For mutants containing single copy insertion, the flanking sequences of the selection marker were determined. Several potential insertion sites were evaluated to be feasible for the insertion of single chain antibody gene.

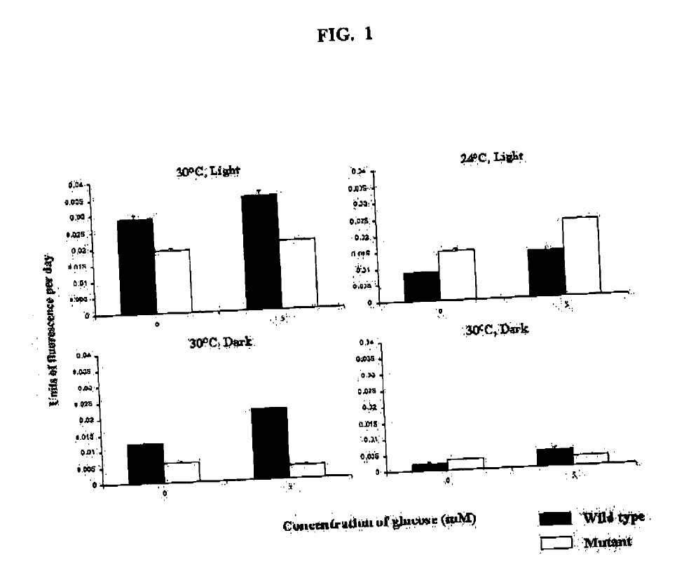

Now referring to the drawing, FIG. 1 shows the growth performance of the rfbJ site knockout mutant for Synechocystis PCC 6803 as compared with that of the wild type in several growth conditions.

Determine the Length of Flanking Sequences that Facilitates Homologous Recombination at a Particular Site:

After determining the insertion site, recombinant DNA molecules containing various length of the flanking sequence and the selection marker were constructed and used for transformation. The shortest flanking sequences that facilitated site-specific insertion were determined. For example, the rfbJ site of Synechocystis PCC 6803, the required flanking sequence was about >500 base pairs for both directions. For example, the rbcS2 site of Chlamydomonas reinhardtii, the required flanking sequence was about >900 base pairs for both directions.

Selection of Native Promoters for High-Level Expression:

The promoter for Km was deleted. The Km without promoter was randomly inserted into the genome of Synechocystis PCC 6803. Mutants with high Km expression were selected on medium containing kanamycin. TAIL-PCR using primer of SEQ ID NO:11 determined promoter driving the expression of Km in each mutant. The function of the native promoter was confirmed by testing its ability to drive the expression of other reporter gene. Equivalent experiments were carried out for Chlamydomonas reinhardtii using Ble T1 and Ble T2 primer.

Determining the Codon Usage Preferences:

One skilled in the art will appreciate that analysis of the preferred codon usage for each species using the published gene sequence information. The codons of single chain antibody gene and reporter genes were modified accordingly.

Screening for Motifs that Facilitate the Expression and Accumulation of Foreign Protein Gene in Algal Cells:

Each motif was inserted at the proper site according to the potential effect of each motif to the reporter gene, and the cassette (i.e., an in-frame sequence) was used for site-specific insertion. Its effect to the expression of reporter gene was evaluated after characterization of the transformant.

The following motifs were determined to be beneficial for foreign gene expression. For Synechocystis PCC 6803, Cmyc is neural and (His)₆ tag is negative. For Chlamydomonas reinhardtii RbcS2 promoter (Goldschmidt-Clermont et al, 1986), TMV omega sequence (Schmitz et al. 1996) linked to the 5′ end, the first intron of RbcS2 (Lumbreras et al. 1998), and the ER retention sequence KDEL (Napier et al. 1992), facilitate foreign gene expression Cmyc and (His)₆ tag are neutral. The above are detailed in Goldschmidt-Clermont, M., and Rahire, M. (1986) J. Mol. Biology 191, 421-432; Lumbreras, V., Stevens, D. R., and Purton, S., (1998) The Plant J 14, 441-447; Napier, R. M., Fowke, L. C., Hawes, C., Lewis, M., and Pelham, H. R. B, (1992) J Cell Sci. 102, 261-271; and Schmitz, J., Prufer, D., Rohde W., and Tacke, E., (1996) Nucl Acids Res 24, 257-263, the teachings of which ae-incorporated herein by reference.

Construct Used for the Transformation of Synechocystis PCC 6803:

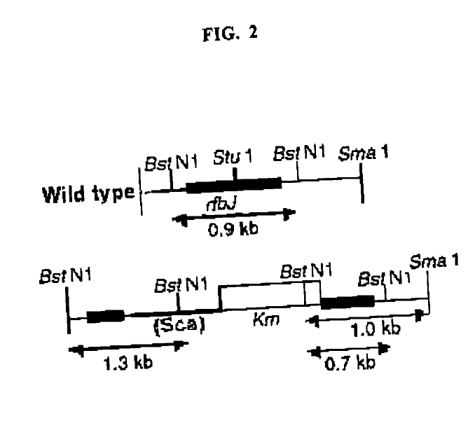

FIG. 2 shows the construct and motifs used for the transformation of Synechocystis PCC 6803.

Construct Used for the Transformation of Chlamydomonas reinhardtii:

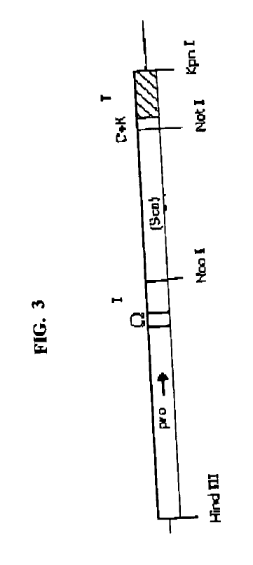

FIG. 3 shows the diagram and motifs used for the transformation of Chlamydomonas reinhardtii.

Definitions:

The terms listed below, as used herein, will have the meaning indicated

Ble=zeomycin resistant gene

(SEQ ID NO: 6)

BleT1 = 5′CATGCCATGGCCAAGCTGACCAGCG-3′

(SEQ ID NO: 7)

BleT2 = 5′CATGCTAGGCGGCCGCGTCCTGCTCCTCGGCCACG3′

BIA=biomolecular interaction analysis
DNA=deoxyribonucleic acid
ER=endoplasmic reticulum
Gfp=green fluorence protein

Km=kanamycin resistant gene

| | |
|---|---|
| KmT1 = 5′CTTCTATCGCCTTCTTGACGA3′ | (SEQ ID NO: 11) |
| KmT2 = 5′CAGAGCAGCCGATTGTCTGTTG3′ | (SEQ ID NO: 12) |

PCR=Polymerase Chain Reaction

RbcS=small subunit of ribulose bisphosphate carboxylase/oxygenase

(SEQ ID NO: 8)

RbcS 2F = 5′-GGTGCCCTCCTGATAAAC-3′

(SEQ ID NO: 9)

RbcS 2B = 5′-AATCCTTTCCTGGAGCCTC-3′

(SEQ ID NO: 10)

RbcS 2INTRON1F = 5′-CACTCAACATCTTAAAATGG-3′

(SEQ ID NO: 13)

RbcLF = 5′ATACGGTCGTCCTCTGCTT3′

(SEQ ID NO: 14)

RbcLB = 5′CCCCTTCCAATTTACCAACC3′

(SEQ ID NO: 1)

RfbJF = 5′CTATCGTTTGGCGGTGCTAGT3′

(SEQ ID NO: 2)

RfbJB = 5′CTATCGTTTGGCGGTGCTAGTTC3′

(SEQ ID NO: 3)

RfbjF100 = 5′TGGGAGTCCAACCGTTGTTAG3′

RT-PCR=reverse transcription-polymerase chain reaction

Sca=single chain antibody

| | |
|---|---|
| ScaF = Sca forward, | |
| 5′AAGTGGATGGGCTGGATAAAC3′ | (SEQ ID NO: 4) |
| ScaB = Sca backward, | |
| 5′GGAAGATGGATACAGTTGGTG3′ | (SEQ ID NO: 5) |

TAIL-PCR=Thermal asymmetric interlaced PCR
TMV=Tobacco Mosaic Virus

### EXAMPLE

The following example demonstrates the expression of a single chain anitibody for progesterone Synechocystis PCC 6803 and Chlamydomonas reinhardtii.

Materials and Methods

1. Constructs Used for Transformation

Coding region for the single chain antibody for progesterone was synthesized based on the published sequence (He et al. 1995). He, M., Kang, A. S., Hamon, M., Humphreys, A. S., Gani, M., and Taussig, M. J., (1995) Immunol. 84, 662-668, the teachings of which are incorporated herein by reference.

The initiation codon was modified from TGT to ATG; and incorporated optimal codon usage for each species. The coding region was inserted into the insertion cloning sites of the DNA molecules illustrated in FIG. 2 and FIG. 3 respectively. Each construct was used for transformation and the transformants were selected. FIG. 2 is a scanned diagram of the recombinant DNA molecule used for Synechocystis PCC 6803 transformation. FIG. 3 is a diagram of the recombinant DNA molecule used for transformation of Chlamydomonas reinhardtii. Pro is RbcS2 promoter, I is RbcS2 intron 1, Sca is the coding region for single chain antibody, C+K is c-Myc+KDEL, T is RbcS2 terminator.

2. Purification of Sca from Algal Extract

Algal cells were broken in buffer containing 20 mM MES/NaOH (pH 6.5), 5 mM MgCl₂, 5 mM CaCl₂, 20% glycerol (v/V), 1 mM PMSF and 5 mM benzamide. Sca was purified from supernatant by protein L affinity resin (Pierce) according to the manufacturer's instruction.

3. Determine the Association Constant and Specificity of Sca Expressed by Algal Cells

Kinetics experiments were conducted using BIAcore 2000. The amino-coupling method was used for immobilizing ligand onto the chip. The Sca was captured by the immobilized progesterone-DSA. The flow rate was 30 μl/min. The rate constants were obtained by averaging 3 replicates measurements. The control experiments were done using blank chip for non-specific binding.

The results were as follows:

1. Confirmation of Correct Insertion at the Target Site

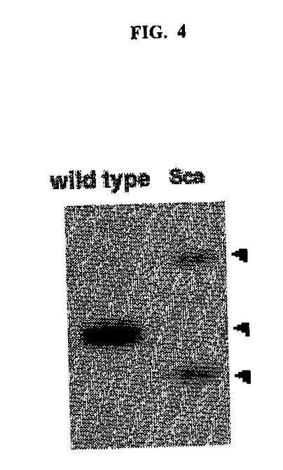

PCR using the primer pairs of SEQ ID NO: 1 and SEQ ID NO: 5 for Synechocystis PCC 6803 and primers of SEQ ID NO: 8 and SEQ ID NO: 5 for Chlamydomonas reinhardtii were used to detect site-specific insertion. DNA sequence of each PCR amplified segment was determined. Further confirmation of single insertion was carried out by genomic Southern blot using the Sca coding sequence as probe. FIG. 4 shows an example of the Southern blot result. The Southern blot result of the transgenic alga that contains a single insert of Sca at the rfbJ target site. The coding region of rfbJ was used as probe.

2. Confirmation of Transcription and Expression of the Inserted Sca

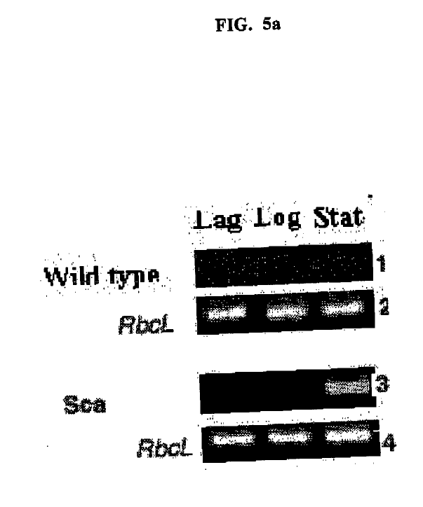

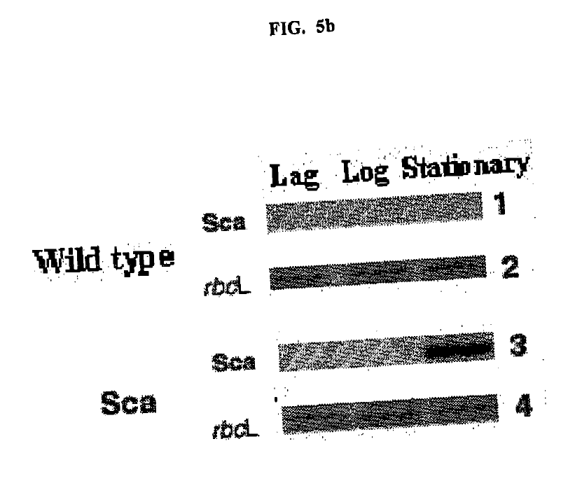

The transcription of Sca in the transgenic algal cells was detected by RT-PCR with total RNA isolated from the transformants. Parallel controls were performed using total RNA isolated from the wild type (FIG. 5a). The RT-PCR result shows the transcription of introduced Sca gene at different stages, the lag phase, log phase and stationary phase, of the growth cycle. All algal cells were grown at 30° C. under the light intensity of 28 μE m⁻² sec^{-1.} The top panel shows the result from the wild type and the bottom panel shows the result from transformant with inserted Sca. For each panel, simultaneous RT-PCR of RbcL gene with primer pair of SEQ ID NO: 13 and SEQ ID NO: 14 were performed for control (2 and 4). Sca transcript was detected with primer pair of SEQ ID NO: 4 and SEQ ID NO: 5 (1 and 3).

The translation and accumulation of Sca in the transgenic algal cells was detected by Western blot analyses using rabbit anti mouse IgG (Fab) that was detected with goat anti rabbit alkaline phosphatase conjugated IgG. Also investigated was the growth condition that induced the highest level of Sca expression. The result is illustrated in FIG. 5b. The Western blot result shows the expression of Sca in different phase of the growth cycle. The algal growth condition was the same as FIG. 5a. Panels 2 and 4 were detected with rbcL antibody and Panels 1 and 3 were detected with rabbit anti-mouse IgG antibody as described above.

3. Functionality and Specificity of the Single Chain Antibody Produced by Transgenic Algae

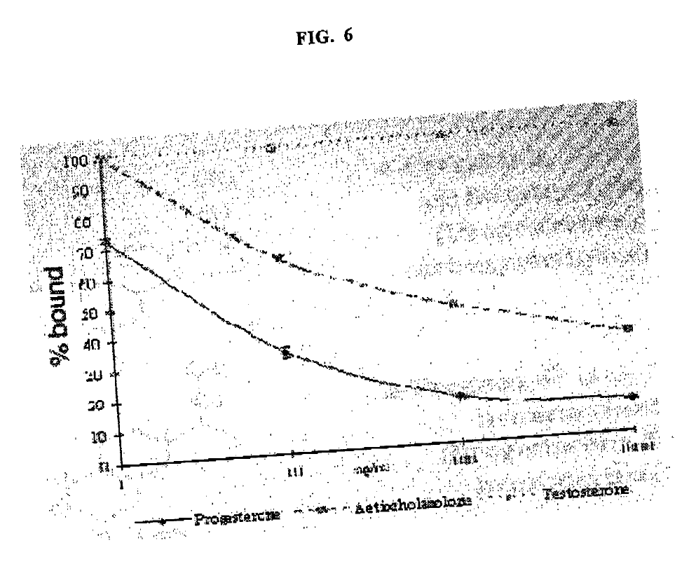

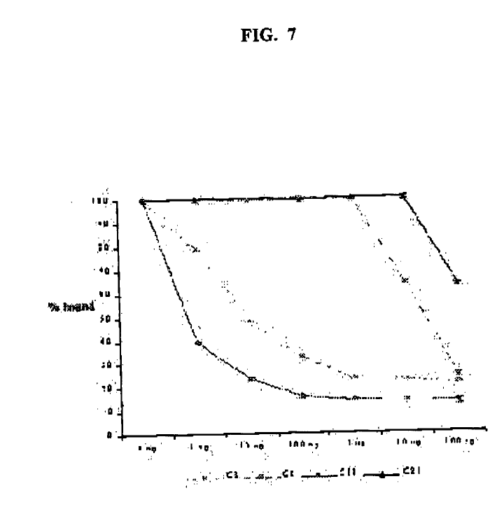

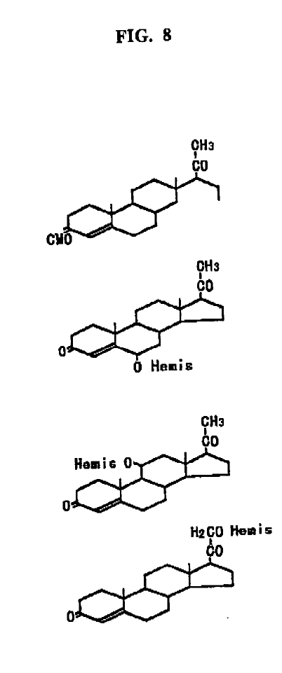

FIG. 6 and FIG. 7 show the specificity of Sca expressed in algal cells and the relative binding affinities for progesterone conjugates, respectively. This demonstrates the Sca produced by algae has high and specific affinity to progesterone and low affinity to testosterone, aetiocholanolone and several progesterone conjugates. FIG. 8 illustrates the structure of the progesterone conjugates used for specificity analysis; for example, 4-Pregnen 3, 20 dione 3 O-carboxymethyloxime (Progesterone 3 carboxymethyloxime) (C₃); 4-Pregnen 6^{β} ol 3, 20 dione hemisuccinate (Progesterone 6^{β} ol hemisuccinate) (C₆); 4-Pregnen 11^{α} ol-3,2-dione Hemisuccinate (Progesterone-11^{α}-ol-Heminsuccinate) (C₁₁); 4-Pregnen-2l ol 3, 20 dione Hemisuccinate (Progesterone-2l-Hemisuccinate) (C₂₁).

4. Yield of Single Chain Antibody Produced by Transgenic Algae

The optimal level can reach about 16% of total algal soluble proteins.

Those skilled in the art will appreciate that many widely different embodiments of the present invention may be adopted without departing from the spirit and scope of the invention.

1. A process for treating pathogen comprising, cloning and expressing a recombinant single chain antibody in heterotrophic algal cells selected from the group consisting of a prokaryotic strain and a eukaryotic strain.

2. The process of claim 1 wherein aquaculture pathogen is controlled.

3. The process of claim 1 wherein agriculture pathogen is controlled.

4. The process of claim 2 or claim 3 wherein the heterotrophic algal cells is the genetically altered prokaryotic strain of Synechocystis PCC 6803.

5. The process of claim 2 or claim 3 wherein the heterotrophic algal cells is the genetically altered prokaryotic strain of Chlamydomonas reinhardtii.

6. An insertion site and Dative promoter combination suitable for foreign gene integration in a heterotrophic prokaryotic strain of Synechocystis PCC 6803.

7. An insertion site and native promoter combination suitable for foreign gene integration in a heterotrophic prokaryotic strain of Chlamydomonas reinhardtii.

8. A genetically engineered DNA molecule that facilitates the site-specific integration and expression of a single chain antibody gene in a prokaryotic alga, Synechocystis PCC 6803, wherein the DNA molecule comprises:
a) a 2.4 kb DNA fragment containing 0.9 kb rfbJ coding region and 1.5 kb upstream and downstream flanking region amplified by using primer pair of SEQ ID NO: 15 and SEQ ID NO: 16;
b) a 0.5 kb promoter region upstream of a psbA II gene amplified by using primer pair of SEQ ID NO: 17 and SEQ ID NO: 18;
c) a 0.5 kb region downstream of the psbA II gene amplified by using primer pair of SEQ ID NO: 19 and SEQ ID NO: 20;
d) the genetic sequences coding for a single chain antibody designed by computational methodology, so as to optimize their acceptability by Synechocystis PCC 6803;
e) the insertion of (b) and (c) at the 5′ end and 3′ end of (d) respectively; and
f) the insertion of (e) at the Stu I site of (a).

9. The genetically engineered DNA molecule of claim 8 wherein the DNA molecule has:
a) 900 base pairs of the rfbJ gene;
b) 500 base pairs of psbA II gene promoter;
c) coding region for a single chain antibody with modified codons; and
d) the stop codon of psbA II gene.

10. A genetically engineered DNA molecule that facilitates the site-specific integration and expression of single chain antibody genes in an eukaryotic alga, Chlamydomonas reinhardtii, wherein the DNA molecule comprises:
a) a 2.7 kb DNA fragment containing 1.07 kb RbcS2 promoter, coding region, 0.22 kb RbcS2 terminator and flanking regions was amplified by using primer pair of SEQ ID NO: 21 and SEQ ID NO: 22;
b) the genetic sequences coding for a single chain antibody designed by computational methodology so as to optimize their acceptability by Chlamydomonas reinhardtii;
c) an omega sequence generated using primer set of SEQ ID NO: 23 and SEQ ID NO: 24;
d) (c) inserted after the promoter region of (a);
e) the region after first intron in (d) replaced by (b);
f) a c-myc and an endoplasmic reticulum retention peptide sequences generated by using the primer set of SEQ ID NO: 25 and SEQ ID NO: 26; and
g) (f) inserted before the RbcS2 terminator in (e).

11. The genetically engineered DNA molecule of claim 10 comprising
a) 1.07 kb region of the RbcS2 including the promoter and first intron;
b) an omega sequence;
c) coding region for a single chain antibody with modified codons;
d) an endoplasmic reticulum retention peptide; and
e) a stop codon of RboS2 gene.

12. Transgenic Synechocystis PCC 6803 heterotrophic strains containing genetically engineered DNA molecule of claim 8 or claim 9 and expresses functional single chain antibody.

13. Transgenic Chlamydomonas reinhardtii heterotrophic strains containing genetically engineered DNA molecule of claim 10 or claim 11 and expresses functional single chain antibody.
